(19) United States
(12) Patent Application Publication
(10) Pub. No.: US 2022/0233523 A1
(43) Pub. Date: Jul. 28, 2022

(54) ANTIBIOTIC POTENTIATION FOR NONTUBERCULOUS MYCOBACTERIAL DISEASE

(71) Applicants: EnBiotix, Inc., Boston, MA (US); Oregon State University, Corvallis, OR (US)

(72) Inventors: Diane M. JOSEPH-MCCARTHY, Boston, MA (US); Luiz BERMUDEZ, Corvallis, OR (US)

(21) Appl. No.: 17/617,711

(22) PCT Filed: Jun. 15, 2020
(86) PCT No.: PCT/US20/37759

§ 371 Date: Dec. 9, 2021

Related U.S. Application Data
(60) Provisional application No. 62/860,990, filed on Jun. 13, 2019.

(51) Int. Cl. A61K 31/496; A61K 9/00; A61K 47/10; A61K 47/69; A61K 31/7036; A61K 31/7048; A61K 9/127; A61K 45/06

(52) U.S. Cl. CPC A61K 31/496; A61K 9/0078; A61K 47/10; A61K 45/06; A61K 31/7036; A61K 31/7048; A61K 9/127; A61K 47/6911

(57) ABSTRACT

The present invention relates to methods and compositions for the treatment of nontuberculous mycobacterium (NTM) infection.

### PRIORITY

This application claims priority to, and the benefit of, U.S. Provisional Application No. 62/860,990 filed Jun. 13, 2019, which is hereby incorporated by reference in its entirety.

### FIELD OF THE INVENTION

The invention relates to methods and compositions for the treatment of nontuberculous mycobacterial infections, and particularly infections of the lung. The present invention provides antibiotic potentiator compositions.

### BACKGROUND

Nontuberculous mycobacterial (NTM) lung disease is a disorder characterized by infection of mycobacteria, particularly mycobacterial species that do not cause tuberculosis or leprosy. NTM are acquired from the environment, and are often found in the water and soil. These organisms commonly affect people with an underlying lung disease such as chronic obstructive pulmonary disease (COPD), bronchiectasis, cystic fibrosis, asthma, primary ciliary dyskinesia, and alpha-1-antitrypsin disease; but individuals with no prior history of lung disease can also be affected. The most common symptoms include a persistent cough, fatigue, weight loss, night sweats, and occasionally shortness of breath and coughing up of blood (hemoptysis). Affected individuals may experience recurrent respiratory infections, which can cause progressive damage to the lungs.

Current treatments generally include antibiotic combinations, such as treatment with one or more of aminoglycoside (e.g., amikacin or streptomycin), macrolide (e.g., azithromycin or clarithromycin), ethambutol, and rifampin, among others. The treatment often continues for 18 months of more, and the treatment often fails to fully eliminate the infection. Ryu Y J, et al., Diagnosis and Treatment of Nontuberculous Mycobacterial Lung Disease: Clinicians' Perspectives, Tuberc. Respir. Dis. 2016 April; 79(2): 74-84. Further, current antibiotic regimens for NTM carry the risk of significant toxicity.

Accordingly, improved and/or alternative therapies for treating NTM are needed.

### SUMMARY

Mycobacterial biofilms favor the survival of bacteria during antibiotic treatment and biofilms are critical for the establishment of infection in vivo. Many protective mechanisms could explain the bacteria's ability to survive antibiotics, including the formation of drug (antibiotic)-tolerant cells, also known as, persister cells. The persister cells are often harbored in biofilms and the presence of such drug-tolerant cells might result in the relapse of persistent bacterial infections after treatment.

The present invention, in various aspects and embodiments, provides methods and compositions (including unit doses) for treating NTM infection in a patient. By improving the potency of an antibiotic regimen and/or avoiding the generation of antibiotic tolerance, the methods and compositions disclosed herein may clear or control NTM infection substantially faster than conventional therapies.

In some aspects, the invention comprises administering to the patient one or more antibiotics, and administering a potentiator composition to the lungs of the patient. In various embodiments, the potentiator composition comprises one or more metabolites selected from metabolites of the Kreb's cycle, a metabolite of the β-oxidation pathway, a metabolite of lipid catabolism, an alkanoic acid or alkanoate, and glycerol. In accordance with embodiments of the invention, an antibiotic-potentiating amount of the metabolite(s) are delivered to anatomical sites of bacterial infection/colonization through inhalation of potentiator into the lung, optionally as a co-formulation with an antibiotic, such as, an aminoglycoside (e.g., amikacin or tobramycin). In the various embodiments, substantial metabolite reaches local sites of infection (including NTM that invade and persist in phagocytic cells) and penetrates mucosal biofilms and is available in the lung epithelial lining fluid to potentiate antibiotic action. The potentiator compounds include carbon substrates that are used by NTM within biofilms, and in nutrient-limited environments.

In some embodiments, the potentiator composition comprises an aliphatic mono- or di-carboxylic acid, or a salt or ester thereof. In some embodiments, the aliphatic mono- or di-carboxylic acid is a straight or branched chain fatty acid, or a salt or ester thereof. In exemplary embodiments, the potentiator composition comprises one or more of: propanoic acid, or salt or ester thereof; butanoic acid, or salt or ester thereof; 2-methylpropanoic acid, or salt or ester thereof; pentanoic acid, or salt or ester thereof; 3-methylbutanoic acid, or salt of ester thereof; caproic acid, 4-methylpentanoic acid, or salt or ester thereof; sebacic acid, or salt or ester thereof; and pyruvic acid, or salt or ester thereof.

Alternatively or in addition, the potentiator composition comprises glycerol and/or acetic acid. Alternatively or in addition, the potentiator composition comprises aliphatic emulsifier compounds that can be used as carbon substrates by biofilm NTM microorganisms, including polysorbates. Exemplary polysorbates include polysorbate 20 (TWEEN 20), polysorbate 40 (TWEEN 40), polysorbate 60 (TWEEN 60), or polysorbate 80 (TWEEN 80).

In various embodiments, the potentiator composition may be administered as an inhaled powder or aerosol. In various embodiments, the potentiator composition is administered by nebulizer. In some embodiments, the potentiator composition comprises liposomes or emulsions, which may contain the aliphatic potentiator compounds described herein. The potentiator composition is effective to potentiate antibiotics that are co-formulated, or administered separately, including orally or by i.v.

In various embodiments, the patient is administered one or more antibiotics, such as one or more selected from: an aminoglycoside antibiotic, a macrolide antibiotic, ethambutol, and a rifamycin. In some embodiments, the aminoglycoside (e.g., amikacin) is administered locally to the lungs, and is optionally a powder formulation or nebulized formulation. For example, the potentiator composition may be a liposomal formulation comprising amikacin and the potentiator compounds, such as the aliphatic potentiator compounds described herein, and/or glycerol and/or acetic acid.

In some embodiments, the patient is administered a macrolide antibiotic, such as azithromycin or clarithromycin.

In various embodiments, a unit dose of the potentiator composition and/or the antibiotic therapy is administered at least three times weekly. In some embodiments, a unit dose (as described herein) of the potentiator composition, and/or the antibiotic therapy is administered once or twice daily. In some embodiments, the administration of the potentiator composition allows for the administration period to be about one year or less, or about nine months or less, or about six months or less. That is, by improving the potency of the antibiotic therapy and/or avoiding the generation of antibiotic tolerant bacteria, the methods and compositions disclosed herein an clear the NTM infection substantially faster than conventional therapies.

In some embodiments, the antibiotic or a salt thereof is formulated as an aqueous solution or suspension or emulsion delivered by a nebulizer. In some embodiments, the formulation is a liposomal formulation of an aminoglycoside antibiotic or salt thereof (e.g., amikacin) and one or more aliphatic potentiators, which can be delivered using a nebulizer. In various embodiments, the methods and compositions provide for delivery of the aminoglycoside antibiotic and an effective amount of the potentiator(s) to distal conducting airways, including in patients with chronic NTM lung disease, in which these distal conducting airways are likely to harbor persistent infection.

In various embodiments, the subject has a non-tuberculous mycobacterial infection involving M. avium, M. avium subsp. hominissuis (MAH), M. abscessus, M. avium complex (MAC) (M. avium and M. intracellulare), or others. In some embodiments, the NTM infection is chronic or recurring. For example, in some embodiments, a prior antibiotic regimen without aminoglycoside (applied for at least about 6 months) was not effective to eradicate or control the infection.

Other aspects and embodiments of the invention will be apparent from the following detailed description.

### BRIEF DESCRIPTION OF DRAWINGS

FIGS. 1A-B show that M. avium in biofilms exhibit a lower capacity to metabolize carbon substrates. Planktonic (white bar) and biofilm (black bar) cultures of M. avium were tested for their capacity to utilize the metabolic substrates available in the Biolog PM1 (FIG. 1A) and PM2A (FIG. 1B) phenotype microarray plate. Both cultures were incubated in PM1 and PM2A plates for 7 days at 37° C. in 100 μl of Biolog inoculating fluid (GN/GP-IF-0a), supplemented with the appropriate additives plus 1× Biolog Redox Dye Mix G. The experiments were performed as an end-point assay. Data represent the means±standard deviations (SD) of the results of 3 experiments performed. *, P<0.05 was considered as statistically significant.

FIGS. 2A-C show that fatty acids promote growth of planktonic M. avium cells. Mycobacteria was cultivated at 37° C. for 12 days, under agitation, in 7H9 broth supplemented with glycerol (FIG. 2C), propionic acid (FIG. 2B), butyric acid (FIG. 2A) (dotted lines). For this assay, we did not supplement 7H9 media with tween (20 or 80), glycerol or OADC (oleic acid, albumin, dextrose and catalase). As a negative control, M. avium was cultivated only in 7H9 broth without any supplementation (solid line). Data represent the means±standard deviations (SD) of the results of 4 experiments performed in triplicate. *, P<0.05 was considered as statistically significant.

FIGS. 3A-D show short chain fatty acids and glycerol affect M. avium biofilm formation. Biofilm formation was performed by seeding 100 μl of mycobacteria suspension made in 7H9 broth containing 1×10⁸ bacteria/ml in 96 wells polystyrene plates. M. avium 104 static biofilms were formed at 37° C. for 7 days and then evaluated through crystal violet methodology. The assays were made in 7H9 media supplemented or not with propionic acid (FIG. 3A) butyric acid (FIG. 3B), caproic acid (FIG. 3C), and glycerol (FIG. 3D). For the current experiment, 7H9 media was not supplemented with tween (20 or 80), glycerol, or OADC. Data represent the means±standard deviations (SD) of the results of 3 experiments performed with eight technical replicates. FIGS. 3A-C**, M. avium biofilms incubated with 1% of fatty acids (propionic acid, butyric acid and caproic acid), as well as with 0.5%, displayed a significant decrease (P<0.05%) in comparison with the M. avium incubation with other concentrations. FIG. 3D* P<0.05 was considered as statistically significant.

FIGS. 4A-D shows that incubation with glycerol, butyric, propionic and caproic acids increase the killing capacity of clarithromycin. Established M. avium 104 biofilms in 96 wells polystyrene plates were incubated for 72 hours with 7H9 only, 7H9 supplemented with metabolite, 7H9 plus clarithromycin and 7H9 supplemented with metabolite plus clarithromycin. FIG. 4A shows data for propionic acid. FIG. 4B shows data for butyric acid. FIG. 4C shows data for caproic acid. FIG. 4D shows data for glycerol. Wells were then mixed 50 times via pipetting to remove attached bacteria and samples were diluted and the colony forming units were obtained to know the total number of viable bacteria. Data represent the means±standard deviations (SD) of the results of experiments performed in quadruplicate. *, P<0.05 was considered as statistically significant.

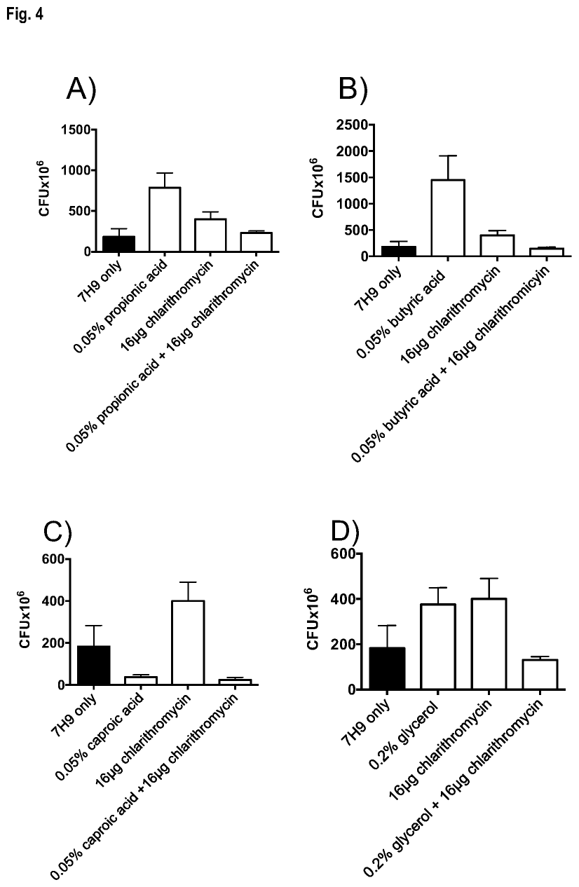

### DETAILED DESCRIPTION OF THE INVENTION

The present invention provides methods and compositions for treating or preventing bacterial infection in the lungs of a subject, and particularly for controlling or eliminating NTM infection in the lungs of a patient. By improving the potency of an antibiotic regimen and/or avoiding the generation of antibiotic resistance, the methods and compositions disclosed herein clear or control NTM infection substantially faster than conventional therapies.

Treatment with antibiotics can induce a persister or drug-tolerant bacterial phenotype, where bacterial cells enter a metabolically dormant state in which bacterial cells are resistant to (or tolerant of) the antibiotics. Thus, the antibiotic helps control, but does not always eradicate chronic infection. The clinical impact of numerous antibiotics are diminished due to this induced bacterial tolerance. The persister or drug-tolerant cells are often harbored in biofilms and the presence of such drug-tolerant cells might result in the relapse of persistent bacterial infections after treatment.

Further, NTM can grow and survive intra-cellularly inside macrophages, which may in part drive drug tolerance. For example, NTM invade the mucosa and get phagocytized by macrophages, where NTM can exhibit robust growth within phagocytic vacuoles. Further, it is believed that NTM persisters develop inside lung lesions as well as within mucus and biofilms. Compounds or compositions that potentiate antibiotic killing of bacteria within macrophages would be of immense value.

Exemplary infections of NTM may involve various non-tubercular mycobacterium species, such as M. avium, M. avium subsp. hominissuis (MAH), M. abscessus, M. chelonae, M. bolletii, M. kansasii, M. ulcerans, M. avium complex (MAC) (M. avium and M. intracellulare), M. chimaera, M. conspicuum, M. peregrinum, M. immunogenum, M. xenopi, M. marinum, M. malmoense, M. mucogenicum, M. nonchromogenicum, M. scrofulaceum, M. simiae, M. smegmatis, M. szulgai, M. terrae, M. terrae complex, M. haemophilum, M. genavense, M. gordonae, M. fortuitum, M. fortuitum complex (M. fortuitum and M. chelonae), or a combination thereof. In some embodiments, the patient has an infection of M. avian complex (MAC).

In some embodiments, the patient has an underlying chronic lung condition, such as cystic fibrosis (CF), non-cystic fibrosis bronchiectasis (non-CFBE), chronic obstructive pulmonary disorder (COPD), asthma, among others, which is exacerbated by NTM infection, presenting risk of substantial pulmonary damage or decline. In some embodiments, the patient has received conventional antibiotic therapy (e.g., macrolide therapy in combination with rifampin and/or ethambutol) for NTM infection, and which failed to fully clear the infection after at least six months.

In some aspects, the invention provides a method for treating NTM infection in the lungs of a patient. The method comprises administering to the patient one or more antibiotics, and administering a potentiator composition to the lungs of the patient. In various embodiments, the potentiator composition comprises one or more metabolites selected from metabolites of the Kreb's cycle, a metabolite of the β-oxidation pathway, a metabolite of lipid catabolism, an alkanoic acid or alkanoate, and glycerol, among others.

In accordance with embodiments of the invention, an antibiotic-potentiating amount of the metabolite(s) are delivered to anatomical sites of bacterial infection/colonization through inhalation of potentiator into the lung, optionally as a co-formulation with an antibiotic suitable for pulmonary delivery, such as, an aminoglycoside (e.g., amikacin or tobramycin). In the various embodiments, substantial metabolite reaches local sites of infection (including NTM that invade and persist in phagocytic cells) and penetrates mucosal biofilms and is available in the lung epithelial lining fluid to potentiate antibiotic action. The potentiator compounds include carbon substrates that are used by NTM within biofilms, and in nutrient-limited environments.

In some embodiments, the potentiator composition comprises an aliphatic mono- or di-carboxylic acid, or a salt or ester thereof. In some embodiments, the aliphatic mono- or di-carboxylic acid, or salt or ester thereof, comprises up to 16 carbon atoms, or comprises up to 10 carbon atoms. In some embodiments, the aliphatic mono- or di-carboxylic acid is a straight or branched chain fatty acid, or a salt or ester thereof. The straight or branched chain fatty acid may be a short chain fatty acid, or a salt or ester thereof. In some embodiments in which a potentiator compound is an ester, the potentiator may be an alkyl ester, such as a methyl or ethyl ester.

In exemplary embodiments, the potentiator composition comprises one or more of: propanoic acid, or salt or ester thereof; butanoic acid, or salt or ester thereof; 2-methylpropanoic acid, or salt or ester thereof; pentanoic acid, or salt or ester thereof; 3-methylbutanoic acid, or salt of ester thereof; caproic acid, 4-methylpentanoic acid, or salt or ester thereof; sebacic acid, or salt or ester thereof; and pyruvic acid, or salt or ester thereof.

Alternatively or in addition, the potentiator composition comprises glycerol and/or acetic acid. Alternatively or in addition, the potentiator composition comprises aliphatic emulsifier compounds that can be used as carbon substrates by biofilm NTM microorganisms, including polysorbates. Exemplary polysorbates include polysorbate 20 (TWEEN 20), polysorbate 40 (TWEEN 40), polysorbate 60 (TWEEN 60), or polysorbate 80 (TWEEN 80).

In various embodiments, the potentiator composition comprises one or more short chain alkanoates. The term “short chain alkanoate” refers to an aliphatic carboxylic acid, including salts or esters thereof. Short chain alkanoates thus include an aliphatic group, such as an alkyl group. Short chain aliphatic groups (e.g., alkyl groups) include those of from 2 to 6 carbon atoms. In some embodiments, the potentiator composition comprises one or more of propionic acid, butyric acid and caproic acid.

In various embodiments, the potentiator composition is formulated for local administration to the lungs of the patient. For example, the potentiator composition may be administered as an inhaled powder or aerosol. In various embodiments, the potentiator composition is administered by nebulizer. In some embodiments, the potentiator composition comprises liposomes or emulsions, which may contain the aliphatic potentiator compounds described herein. The potentiator composition is effective to potentiate antibiotics that are co-formulated, or administered separately, including by inhalation, orally or by i.v.

In various embodiments, the patient is administered one or more antibiotics, such as one or more selected from: an aminoglycoside antibiotic, a macrolide antibiotic, ethambutol, and a rifamycin.

In some embodiments, the patient is administered an aminoglycoside antibiotic selected from amikacin, streptomycin, tobramycin, apramycin, arbekacin, astromicin, capreomycin, dibekacin, framycetin, gentamicin, hygromycin B, isepamicin, kanamycin, neomycin, netilmicin, paromomycin, rhodestreptomycin, ribostamycin, sisomicin, spectinomycin, and verdamicin, or a pharmaceutically acceptable salt thereof. In some embodiments, the patient is administered amikacin or streptomycin or a pharmaceutically acceptable salt thereof.

In some embodiments, the aminoglycoside is administered locally to the lungs, and is optionally a powder formulation or nebulized formulation. For example, in some embodiments, amikacin is administered locally to the lungs, and is contained within the potentiator composition. For example, the potentiator composition may be a liposomal formulation comprising amikacin and the potentiator compounds, such as the aliphatic potentiator compounds described herein, and/or glycerol and/or acetic acid.

In these or other embodiments, the patient is administered a macrolide antibiotic. Exemplary macrolide antibiotics include azithromycin, clarithromycin, erythromycin, fidaxomicin, carbomycin A, josamycin, kitasamycin, midecamycin acetate, oleandomycin, solithromycin, spiramycin, troleandomycin, tylosin, tylocine, and roxithromycin, or a pharmaceutically acceptable salt thereof. In various embodiments, the macrolide is administered orally. In exemplary embodiments, the macrolide is selected from azithromycin or clarithromycin.

In these or other embodiments, the patient is administered a rifampicin, such as rifampin or rifabutin, which may be administered orally. Rifampin, is used to treat several types of bacterial infections, including tuberculosis, Mycobacterium avium complex, leprosy, and Legionnaires' disease. Rifampcin is typically used together with other antibiotics. Rifamicins act by decreasing the production of RNA by bacteria, by inhibiting bacterial DNA-dependent RNA polymerase.

In these or other embodiments, the patient may be administered ethambutol, which may be administered orally. Ethambutol is an antibiotic primarily used to treat tuberculosis and NTM infections. It is usually given in combination with other agents. Ethambutol is bacteriostatic against actively growing bacteria, and acts by obstructing the formation of cell wall.

In some embodiments, the patient receives at least two, three, or four antibiotics, including at least two or three antibiotics disclosed herein. In some embodiments, the patient receives no more than 3 or 2 antibiotic compositions or agents, thereby avoiding some antibiotic toxicity.

In various embodiments, the potentiator composition and/or the antibiotic therapy is administered at least three times weekly or at least five times weekly. In some embodiments, the potentiator composition and/or the antibiotic therapy is administered once or twice daily. In various embodiments, the administration period for the therapy is at least about 6 months, but in some embodiments, is at least about 12 months, or at least about 18 months. In some embodiments, the administration of the potentiator composition allows for the administration period to be about one year or less, or about nine months or less, or about six months or less. That is, by improving the potency of the antibiotic and/or avoiding the generation of antibiotic tolerant bacteria, the methods and compositions disclosed herein clear the NTM infection substantially faster than conventional therapies.

In exemplary embodiments where an antibiotic (e.g., an aminoglycoside such as amikacin or tobramycin) is formulated within the potentiator composition, the antibiotic potentiators may be present at a molar ratio of from about 1000:1 to about 10:1 (potentiator:aminoglycoside), or in some embodiments from about 500:1 to about 10:1, or from about 100:1 to about 10:1, or from about 50:1 to about 10:1. In some embodiments, the aminoglycoside is amikacin or salt thereof, such as amikacin sulfate. In some embodiments, the formulation contains from about 200 to about 800 mg of amikacin of salt thereof per unit dose. In some embodiments, the formulation contains from about 400 to about 600 mg of amikacin or salt thereof per dose (e.g., about 600 mg).

In some embodiments, the antibiotic or a salt thereof is formulated as an aqueous solution or suspension delivered by a nebulizer. In some embodiments, the formulation is a liposomal formulation of the antibiotic or salt thereof and the potentiator. In some embodiments, the formulation is provided at a unit volume in the range of about 5 mL to about 12 mL, and in some embodiments between about 6 mL and about 10 mL.

Various types of nebulizers are known. The type of nebulizer can influence the amount of antibiotic and/or potentiator that reaches sites of infection or colonization. As used herein, the term “nebulizer” refers to a drug delivery device to administer medication in the form of a mist inhaled into the lungs. Nebulizers use oxygen, compressed air, or ultrasonic power to break up solutions and suspensions into small aerosol droplets that can be directly inhaled from the mouthpiece of the device. The lung deposition characteristics and efficacy of an aerosol depend largely on the particle or droplet size; for example, the smaller the particle the greater its chance of peripheral penetration and retention. Particles smaller than about 5 μm in diameter deposit frequently in the lower airways, and therefore are desirable for pharmaceutical aerosols.

In some embodiments, the nebulizer is a Jet nebulizer. Jet nebulizers are connected by tubing to a compressor, which causes compressed air or oxygen to flow at high velocity through a liquid medicine to turn it into an aerosol, which is then inhaled by the patient. In some embodiments, the nebulizer is an ultrasonic wave nebulizer. An ultrasonic wave nebulizer uses an electronic oscillator to generate a high frequency ultrasonic wave, which causes the mechanical vibration of a piezoelectric element. This vibrating element is in contact with a liquid reservoir and its high frequency vibration is sufficient to produce a vapor mist. In some embodiments, the nebulizer involves a vibrating, perforated membrane designed to improve upper and lower respiratory tract deposition of a liposome formulation.

In some embodiments, the potentiator composition is an aqueous solution or suspension or emulsion, delivered with the use of a nebulizer, and which contains the antibiotic or a salt thereof at from about 200 to about 800 mg per unit dose. In some embodiments, the formulation contains from about 400 to about 600 mg of the aminoglycoside (e.g., amikacin or tobramycin) or salt thereof per unit dose. In some embodiments, the invention allows for the aminoglycoside or salt thereof to be delivered at substantially lower unit doses than 600 mg, while having the same or greater efficacy. In some embodiments, the formulation contains antibiotic or salt thereof at from about 200 to about 400 mg per unit dose. Unit doses can be provided in individual ampules.

The bioavailability of the aminoglycoside tobramycin in the lung of cystic fibrosis patients upon local delivery has been the subject of investigation. For example, sputum samples expectorated at 10 minutes after delivery of 300 mg of tobramcyin by nebulizer showed a Mean of 1,237 μg/g of sputum. Geller D E., et al., Pharmacokinetics and Bioavailability of Aerosolized Tobramycin in Cystic Fibrosis, Chest 122(1) (2002). In contrast to expectorated sputum, sputum induction by inhalation of hypertonic saline samples respiratory secretions from more distal conducting airways, which are often sites of infection in CF. Using this sampling process, tobramycin concentrations in the lung epithelial fluid were estimated to be in the range of 128 μg/g, after 300 mg of tobramycin was delivered by nebulizer. Ruddy J, et al., Sputum Tobramycin Concentrations in Cystic Fibrosis Patients with Repeated Administration of Inhaled Tobramycin, J. Aerosol Med. And Pulmon. Drug Del. 26(2): 69-75 (2013).

In various embodiments, the methods and compositions provide for delivery of an aminoglycoside antibiotic and an effective amount of the potentiator to distal conducting airways, including in patients with chronic NTM lung disease, in which these distal conducting airways are likely to harbor persistent infection.

In various embodiments, the nebulizer formulation (i.e., a unit dose) contains from about 400 mg to about 5000 mg per unit dose of the potentiator compounds. In some embodiments, the formulation contains from about 400 to about 2500 mg per dose, or about 400 to about 2000 mg per dose, of the potentiator compounds. In some embodiments, the potentiator and antibiotic are administered in a 2 to 10 mL solution by nebulizer. The metabolite delivered by nebulizer penetrates to areas of infection and/or colonization in sufficient levels to potentiate antibiotic action.

In still other embodiments, the formulation is a dry powder for inhalation. In such embodiments, the unit dose formulation may comprise from about 400 mg to about 5000 mg per unit dose of the potentiator compounds. In some embodiments, the formulation contains from about 400 to about 2500 mg per unit dose, or about 400 to about 2000 mg per unit dose, of the potentiator compounds. In some embodiments, the formulation may contain an antibiotic (e.g., aminoglycoside, such as amikacin or tobramycin) or salt thereof, for example, at about 75 mg to about 200 mg per dose. The powder unit dose formulation may take the form of subdoses, for example, where 2, 3, 4, 5 or more subdoses (e.g., capsules) are administered as a single dose using an inhaler device.

An exemplary inhaler device suitable for delivery of dry powder formulations is TOBI PODHALER (Novartis). For example, a capsule containing a single sub dose is inserted into the capsule chamber of the device, a mouthpiece screwed over the top, the capsule is then pierced and the powder contents inhaled (generally with two breaths). The remaining subdoses are then delivered to constitute a single delivery.

An exemplary delivery system for a liposomal formulation of the antibiotic (such as amikacin), is described, for example, in U.S. Pat. Nos. 10,588,918; 10,398,719; 10,251,900; 10,238,675, which are hereby incorporated by reference in its entirety. The liposomal formulation is a convenient form for incorporating the aliphatic potentiator compound(s), and can facilitate formulation and delivery of a sufficient amount of aliphatic potentiator to sites of bacterial infection. For example, the formulation may comprise the liposomal complexed antibiotic (e.g., aminoglycoside such as amikacin or tobramycin) as a dispersion (e.g., a liposomal solution or suspension). The liposomal portion of the composition may comprise a lipid component that includes electrically neutral lipids, as well as optionally cationic and/or anionic lipids. Exemplary formulations comprise a phosphatidylcholine and a sterol (e.g., dipalmitoylphosphatidylcholine and cholesterol). In various embodiments, upon nebulization, the aerosolized composition has an aerosol mean droplet size of about 1 μm to about 3.8 μm, or about 1.0 μm to about 4.8 μm, or about 3.8 μm to about 4.8 μm, or about 4.0 μm to about 4.5 μm. In some embodiments, the mean droplet size is less than about 5 μm, or less than about 4 μm, or less than about 3 μm. In various embodiments, the phospholipids comprise one or more of a phosphatidylcholine (PC), phosphatidylglycerol (PG), phosphatidylinositol (PI), phosphatidylserine (PS), phosphatidylethanolamine (PE), and phosphatidic acid (PA).

In various embodiments, the subject has a non-tuberculous mycobacterial infection involving M. avium, M. avium subsp. hominissuis (MAH), M. abscessus, M. chelonae, M. bolletii, M. kansasii, M. ulcerans, M. avium complex (MAC) (M. avium and M. intracellulare), M. chimaera, M. conspicuum, M. peregrinum, M. immunogenum, M. xenopi, M. marinum, M. malmoense, M. mucogenicum, M. nonchromogenicum, M. scrofulaceum, M. simiae, M. smegmatis, M. szulgai, M. terrae, M. terrae complex, M. haemophilum, M. genavense, M. gordonae, M. fortuitum, M. fortuitum complex (M. fortuitum and M. chelonae), or a combination thereof. In some embodiments, the NTM infection is chronic or recurring. For example, in some embodiments, a prior antibiotic regimen without aminoglycoside (applied for at least about 6 months) was not effective to eradicate or control the infection.

In some aspects, the invention provides a unit dose formulation for delivery by nebulizer, the formulation comprising: from 100 to 600 mg of an aminoglycoside antibiotic or a salt thereof, and effective amount of an aliphatic mono- or di-carboxylic acid, or a salt or ester thereof, to potentiate the aminoglycoside activity against nontuberculous mycobacterium (NTM). For example, as disclosed herein, the aliphatic mono- or di-carboxylic acid, or salt or ester thereof, may comprise up to 16 carbon atoms, or comprises up to 10 carbon atoms.

In some embodiments, the unit dose formulation comprises an aliphatic mono- or di-carboxylic acid is a straight or branched chain fatty acid, or a salt or ester thereof. The potentiators may include one or more straight or branched chain fatty acid, such as a short chain fatty acid, or a salt or ester thereof. In some embodiments, the short chain fatty acid is provided as an alkyl ester, which is optionally a methyl or ethyl ester. Exemplary potentiator compounds include: propanoic acid, or salt or ester thereof; butanoic acid, or salt or ester thereof; 2-methylpropanoic acid, or salt or ester thereof; pentanoic acid, or salt or ester thereof; 3-methylbutanoic acid, or salt of ester thereof; caproic acid, 4-methylpentanoic acid, or salt or ester thereof; sebacic acid, or salt or ester thereof; and pyruvic acid, or salt or ester thereof.

In these or other embodiments, the unit dose further comprises glycerol. For example, the unit dose may comprise from about 0.5% to about 5% glycerol by weight, such as from about 1% to about 5% glycerol by weight, or from about 2% to about 5% glycerol by weight. Alternatively or in addition, the unit dose further comprises acetic acid.

For example, the unit dose may comprise aminoglycoside antibiotic amikacin, and the amikacin is comprised in liposomes with one or more aliphatic potentiator compounds described herein.

In some embodiments, the unit dose formulation is packaged in ampules of from 5 to 15 mL, or in ampules of from about 5 to about 10 mL.

In some embodiments, the formulation is a liposomal formulation of the antibiotic (such as amikacin), as described, for example, in U.S. Pat. Nos. 10,588,918; 10,398,719; 10,251,900; 10,238,675, which is hereby incorporated by reference in its entirety. For example, the formulation may comprise the liposomal complexed antibiotic (e.g., aminoglycoside such as amikacin or tobramycin) as a dispersion (e.g., a liposomal solution or suspension). The liposomal portion of the composition may comprise a lipid component that includes electrically neutral lipids, as well as optionally cationic and/or anionic lipids. Exemplary formulations comprise a phosphatidylcholine and a sterol (e.g., dipalmitoylphosphatidylcholine and cholesterol). In various embodiments, upon nebulization, the aerosolized composition has an aerosol mean droplet size of about 1 μm to about 3.8 μm, or about 1.0 μm to about 4.8 μm, or about 3.8 μm to about 4.8 μm, or about 4.0 μm to about 4.5 μm. In some embodiments, the mean droplet size is less than about 5 μm, or less than about 4 μm, or less than about 3 μm. In various embodiments, the phospholipids comprise one or more of a phosphatidylcholine (PC), phosphatidylglycerol (PG), phosphatidylinositol (PI), phosphatidylserine (PS), phosphatidylethanolamine (PE), and phosphatidic acid (PA).

Sterols for use with the formulation include, but are not limited to, cholesterol, esters of cholesterol including cholesterol hemi-succinate, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate, lanosterol sulfate and tocopherols. The tocopherols can include tocopherols, esters of tocopherols including tocopherol hemi-succinates, salts of tocopherols including tocopherol hydrogen sulfates and tocopherol sulfates.

In some embodiments, the unit dose formulation may comprise other aliphatic emulsifier compounds that can be used as carbon substrates by biofilm NTM microorganisms, including polysorbates. Exemplary polysorbates include polysorbate 20 (TWEEN 20), polysorbate 40 (TWEEN 40), polysorbate 60 (TWEEN 60), or polysorbate 80 (TWEEN 80).

In some embodiments, the invention provides a unit dose formulation for delivery by nebulizer, the formulation comprising in an aqueous solution or liposomal suspension from 100 to 600 mg of the aminoglycoside antibiotic of salt thereof (e.g., amikacin); and from about 100 mg to about 2000 mg of one or a combination of the potentiators described herein, or in some embodiments, from about 500 to about 1500, or from about 500 to about 1000 mg of one or more potentiators described herein. The formulation may be packaged in unit dose ampules having a volume of from 5 to 15 mL, such as in unit dose ampules of from about 5 to about 10 mL.

In some embodiments, the formulation is delivered to a patient having or at risk of an NTM lung condition. In some embodiments, the patient has a pre-existing chronic lung disease, such as, for example, cystic fibrosis, bronchiectasis, chronic obstructive pulmonary disorder (COPD), idiopathic pulmonary fibrosis, or asthma, among others. In some embodiments, the method and formulation described herein is used for treating an NTM infection of the lung of such patients. In some embodiments, the patient presents with cavitary disease, in which scarring (fibrosis) of cavities are observed in the lungs (cavitation).

Other aspects and embodiments of the invention will be apparent from the following examples.

### EXAMPLES

### Materials and Methods

### Bacteria Culture:

The Mycobacterium avium subsp hominissuis 104 (MAH104), isolated from the blood of AIDS patients, was used in the current study. Mycobacterium was cultivated in 7H10 Middlebrook broth medium (SIGMA) supplemented with 10% OADC (oleic acid, albumin, dextrose and catalase; HARDY DIAGNOSTICS, Santa Maria, Calif.) at 37° C. for 7 days. For some experiments, MAH104 was cultivated in 7H9 medium supplemented with 10% OADC at 37° C. for 7 days.

### Biofilm Formation:

Static biofilms were used in the present study as described previously (Rose S J, Bermudez L E., Infection and Immunity 2014; 82:405-12). Briefly, mycobacteria were taken from 7H10 agar plates and resuspended in deionized water (10⁹ of colony forming units/ml; CFU/ml). The bacterial suspension was then washed 3 times with deionized water to remove any remaining of 7H10 media (3500 rpm for 20 minutes at 20° C.). After washing, the MAH104 were resuspended in 7H9 medium without any carbon sources (OADC, glycerol, tween 20 and tween 80) (non-supplemented 7H9) and suspensions were left alone to allow clumped bacteria to settle. The top half of the suspension was transferred to new tube and adjusted to 1×10⁸ CFU/ml, using visual turbidity and optical density. Suspensions were inoculated in 96-well polystyrene plates (BD, Franklin Lakes, N.J.) (100 μl for each well [or 10⁷ bacteria per well]) and biofilms were formed at 37° C. for 7 days or for 14 days when indicated. Crystal violet was solubilized with 33% of acetic acid and the O.D. at 570 nm was determined (Stepanovic et al., European journal of clinical microbiology & infectious diseases 2001; 20:502-4). The raw O.D. at 570 nm values were discounted from O.D. 570 nm average of blank wells (wells with only 7H9 media).

### Metabolic Phenotype Study of Planktonic and Biofilm MAH104 Cultures:

Metabolic phenotype study was performed with planktonic and biofilm cultures using the 96-well plates PM1 and PM2A (BIOLOG™, Hayward, Calif.). The assays with planktonic cells and biofilms were made at the same time. Each plate contains carbon substrates and one negative control well, in which the bacteria is tested without any substrate. The tests with planktonic and biofilm cultures were performed at the same time and the culture inoculum have the same number of passages. As recommended by BIOLOG, the plates PM1 and PM2A were incubated for 7 days at 37° C. with inoculating media IF-0a GN/GP (1.2×) plus the appropriate PM additives and Biolog Dye G Mix (100×) but without MAH104 to check for abiotic reactions. The experiments were made as an end-point assay.

To prepare the inoculum of planktonic cells for PM plates, MAH104 was cultivated in 7H9 medium with 10% OADC and 0.05% of tween20. After the cells reached a mid-logarithmic phase (O.D. at 595 nm=0.3 to 0.6), the cultures were harvested and washed three times with deionized water (centrifugation conditions; 3500 rpm for 20 minutes at 20° C.) and incubated in water for 24 hours at 25° C. as a starvation step. The bacteria suspension was centrifuged and pellet was resuspended in 1 ml of inoculating media IF-0a GN/GP (1.2×). Part of the resuspended cells were transferred to a new falcon tube with 10 ml of IF-0a GN/GP (1.2×), 120 μl of Biolog Dye G Mix (100×) and with 1 ml of PM additives solution (24 mM magnesium chloride; 12 mM calcium chloride; 0.0012% zinc sulfate; 0.06% ferric ammonium citrate; 1.2% ammonium chloride; 0.01% tween20) until the cells reached 81% of percentage transmittance. A volume of 100 μl of this final bacteria suspension was inoculated into the wells of PM1 and PM2A plates. These plates were incubated for 7 days at 37° C. and after the incubation the O.D. at 590 nm of the plates were measured.

The cultures of biofilms were established for 7 days in regular polystyrene 96 well plates, not in PM Biolog plates, as described above with a minor difference. For this experiment, biofilms were formed in IF-0a GN/GP media (1.2×) without PM additives solution instead of non-supplemented 7H9 media. Once biofilms were established, PM1 and PM2A plates were incubated with 100 μl of bacteria-free IF-0a GN/GP (1.2×) that already contains Biolog Dye G Mix (100×) and PM additives solution for 30 minutes at 37° C. in order to solubilize the metabolites present in the PM plates. The IF-0a GN/GP (1.2×) containing the solubilized substrates was transferred to the wells in which biofilms were formed and incubated for additional 7 days at 37° C. (total of 14 days of biofilm incubated at 37° C.). To obtain spectrophotometric measurements from biofilm cultures (O.D._{590 nm}), the plate was centrifuged for 20 minutes at 3500 rpm at 20° C., which reduce bacteria interference with the readings, and biofilm supernatant was transferred to a new 96 well plates for O.D._{590 nm} measurement.

Effect of Short-Chain Fatty Acids and Glycerol in MAH04 Planktonic Cultures of M. avium:

Planktonic cultures of MAH104 were incubated with propionic acid, butyric acid, and glycerol (all purchased from SIGMA) to determine whether these metabolites interfere with the growth of planktonic cultures in nutrients-limited media (non-supplemented 7H9). MAH104 from 7H10 agar was used to make bacterial suspension in deionized water (10⁹ CFU/ml) and then the suspension was centrifuged three times to wash the bacteria cells (3500 rpm for 20 minutes at 20° C.). After washing step, the bacterial suspensions were resuspended in 7H9 medium and suspensions were left alone to allow clumped bacteria to settle. The top half of the suspension was transferred to new tube and adjusted to 1×10⁸ CFU/ml, using visual turbidity and optical density. The suspensions were inoculated (50 μl/5×10⁶ CFU) into 3 ml of 7H9 with or without different concentrations of propionic acid and butyric acid (1%, 0.5%, 0.1%, 0.05%, 0.01%) and 0.2% of glycerol. These cultures were incubated at 37° C. for 12 days and the O.D._{595 nm} was measured at each 72 hours.

Biofilm Assays with Propionic Acid, Butyric Acid and Glycerol:

Assays with biofilms incubated with propionic acid, butyric acid, and glycerol were performed to test whether these metabolites affect the formation of biofilms. All the experiments were made in non-supplemented 7H9. The effect of these metabolites was evaluated during biofilm formation, in pre-established biofilms and in M. avium cells pre-attached to polystyrene. To evaluate the effect of targeted metabolites during biofilm formation, mycobacteria suspensions were prepared and inoculated in 96 wells polystyrene plates, as already described, and biofilms were formed in the presence or not of the short-chain fatty acids at different concentrations (1%, 0.5%, 0.1%, 0.05% and 0.01%). For glycerol, only 0.2% was used. The biofilms were formed in 7H9 media at 37° C. for 7 days. For pre-established biofilms, MAH104 was again inoculated in 96 wells plates and biofilms were formed as described herein. After seven days, the supernatant of biofilms were discarded to remove planktonic bacteria and established biofilms were incubated with non-supplemented 7H9 with or not with the targeted metabolites (propionic acid, butyric acid, and glycerol) for 7 days at 37° C. It was also evaluated whether these metabolites could promote the growth of MAH104 cells present in biofilms through the measurement of 0.11595 nm every 24 hours. Finally, mycobacteria suspension with low density made in 7H9 broth (containing 1×10⁶ bacteria/ml) were seeded in 96 wells polystyrene plates and incubated for 7 days at 37° C. The supernatant was removed and M. avium cells attached to the plate were incubated with 7H9 supplemented or not with 0.05% propionic acid, 0.05% butyric acid, 0.05% and 0.2% glycerol. The growth of pre-attached bacterial cells was followed every 24 hours through O.D._{595 nm}. The biofilm formation was evaluated by crystal violet.

Treatment of MAH Biofilms with Antibiotics:

To assess the antibiotic tolerance of MAH biofilms to amikacin (4 μg/ml) and clarithromycin (16 μg/ml), biofilms were formed for 14 days as described herein. Subsequently, the supernatants were gently removed and replaced with new non-supplemented 7H9 media containing antibiotics and supplemented with or without the targeted metabolites (0.05% propionic acid, 0.05% butyric acid, or 0.2% glycerol) at 37° C. As a negative control, biofilms were incubated only with non-supplemented 7H9 without any antibiotics. In addition, MAH biofilms were incubated only with non-supplemented 7H9 containing only 0.05% propionic acid, 0.05% butyric acid, or 0.2% glycerol. Subsequently, 100 μl of 0.02% of Triton X-100 (final concentration 0.01%) was added to the established biofilms to mix, dilute and perform CFU analysis with the entire population of biofilms (attached and unattached).

Treatment of MAH inside Human Macrophages with Antibiotics:

Human THP-1 cell line (TIB-202) (American Type Culture Collection, Manassas, Va.) was cultivated in RPMI-1640 medium with 10% of heat-inactivated fetal bovine serum (FBS, GEMINI B10-PRODUCTS, Sacramento, Calif.), at 37° C. with 5% CO₂. The THP-1 cells were maintained in 75 cm² tissue culture flasks. The differentiation of THP-1 monocytes into macrophages with PMA (phorbol 12-myristate 13-acetate) (SIGMA ALDRICH) and intracellular antibiotic killing assays were performed as previously described (Rojony et al., Clinical proteomics 2019; 16:39). After removal of extracellular bacteria through amikacin treatment (400 μg/ml for 1 h), infected monolayers of THP-1 macrophages were treated with either amikacin (4 μg/ml), clarithromycin (16 μg/ml), antibiotic (amikacin or clarithromycin) plus targeted metabolites (0.05% propionic acid, 0.05% butyric acid, or 0.2% glycerol) or only with the targeted metabolites. As a negative control, differentiated THP-1 cells were incubated without any antibiotics and with no metabolites. THP-1 cells were replenished with new media containing antibiotics, or no antibiotic, every other day. Cells were lysed at 2 h (baseline) and day 4 and subsequently the number of viable bacteria was determined by CFU counting on 7H10 agar plates.

### Statistical Analysis:

Unpaired two tailed t test was performed for the statistical comparisons between groups. The statistical analysis and graphical outputs were made in GraphPad Prism software (version 6.0).

### Example 1: M. avium in Biofilms Showed a Lower Capacity to Utilize Carbon Substrates

An initial screen was performed using Biolog PM1 and PM2A Phenotype Microarray plates to determine which carbon substrates are used by MAH104 when in biofilms. The experiment was designed as an endpoint assay at the same time with biofilms and planktonic cultures and then O.D._{590 nm} values obtained for both cultures were compared. The data show that planktonic cultures were able to use 16 out of 190 (8.4%) substrates, while biofilms used 11 substrates (5.7%) (Table 1).

TABLE 1
Metabolites used by plankton and biofilm M. avium
cultures in Biolog plates

| | | | |
|---|---|---|---|
| Metabolite | Planktonic | Biofilm | Abiotic reactions |

PM1 Plate

| | | | |
|---|---|---|---|
| Negative control | − | − | − |
| Glycerol | + | + | − |
| Tween 20 | + | + | − |
| Acetic Acid | + | + | − |
| Tween 40 | + | + | − |
| α-Keto-Glutaric acid | + | − | − |
| α-Keto-Butyric acid | + | − | − |
| Tween 80 | + | + | − |
| α-Hydroxy Butyric | + | − | − |
| acid | | | |
| Propionic acid | + | + | − |
| Acetoacetic acid | + | − | − |
| Mono Methyl | + | + | − |
| Succinate | | | |
| Methyl Pyruvate | + | + | − |
| Pyruvic acid | + | + | − |

PM2A Plate

| | | | |
|---|---|---|---|
| Negative Control | − | − | − |
| Butyric acid | + | + | − |
| Caproic acid | + | + | − |
| Sebacic acid | + | + | − |

Experiments were performed three times as an end-point assay. If planktonic and/or biofilm cultures were able to metabolize a specific metabolite substrate, electrons from NADH reduce the Redox dye in an irreversible reaction that generates a purple color in the PM plate wells. The signal (+) and (−) indicates, respectively, whether a purple color was produced or not. Abiotic reductions of the Redox dye were assessed through adding bacteria-free GN/GP-IF-0a fluid in the wells. Negative control corresponds to the wells without any metabolic substrate. All other metabolic substrates available in PM1 and PM2A plate were not metabolized by plankton or biofilm cultures.

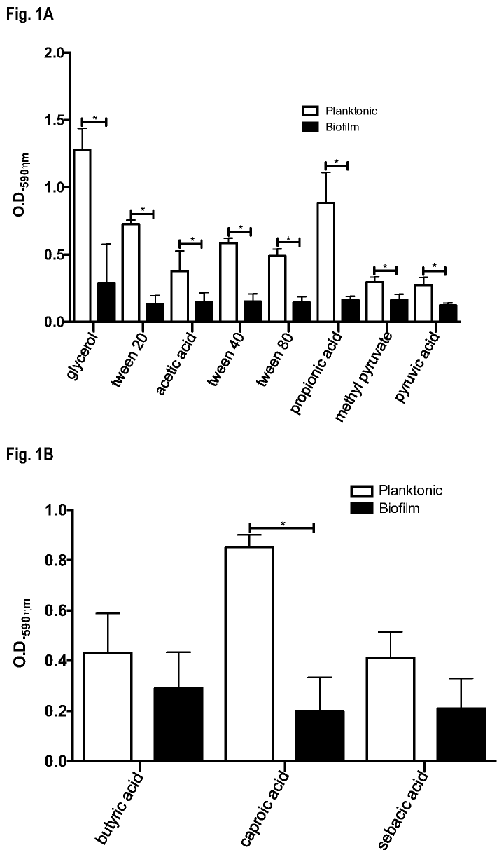

Within the set of metabolites used by planktonic cells, the biofilms were unable to metabolize α-keto glutaric acid, α-keto butyric acid, α-hydroxy butyric acid, acetoacetic acid, and monomethyl succinate. Furthermore, it was demonstrated that MAH104 biofilms presented a significant lower capacity to use glycerol (reduced by 11.14 fold), TWEEN 20 (decrease of 5.4×), TWEEN 40 (3.8-fold lower), acetic acid (2.55× lower), TWEEN 80 (3.45× reduction), propionic acid (a drop of 5.49×), methyl pyruvate (1.8-fold lower), pyruvic acid (2.21× lower) and caproic acid (4.27× reduction) (FIG. 1A, B). No significant difference was found for butyric acid (Biofilm O.D._{590 nm}=0.430±0.091, planktonic O.D._{590 nm}=0.289±0.083, P=0.31) and sebacic acid (Biofilm O.D._{590 nm}=0.411±0.059, planktonic O.D._{590 nm}=0.210±0.069, P=0.092).

### Example 2: Short-chain Fatty Acids and Glycerol Can Support the Growth of Sessile M. avium in Nutrient-limited Media

Further assays were made with the short-chain fatty acids propionic acid and butyric acid, as well as glycerol, to evaluate their effects on MAH104 cells in conditions that are known to induce antibiotic tolerance in MAH104 cells —nutrient-limited media (Anderl et al., Antimicrobial agents and chemotherapy 2003, 47:1251-6; Greendyke et al., Antimicrobial agents and chemotherapy 2008, 52:2019-26; Archuleta et al., Tuberculosis (Edinburgh, Scotland) 2005; 85:147-58) and in biofilms (Rose et al., Structural Integrity, and Tolerance to Antibiotics. PloS one 2015; 10:e0128772). The criteria used to choose these metabolites was to test one metabolite that was highly significantly used by planktonic cells (propionic acid) and another one with no significant differences between planktonic and biofilm cultures (butyric acid). Glycerol was also included in the experiments since it is a well-known compound already used for planktonic cultures. Recent findings revealed that slow growth is directly linked with drug-tolerance (Pontes et al., Science signaling 2019; 12) and some works support the idea that actively dividing cells exhibit a higher susceptibility to antibiotics than non-dividing cells (Anderl et al., Antimicrobial agents and chemotherapy 2003; 47:1251-6; Fux et al., Journal of bacteriology 2004, 186:4486-91; Gradelski et al., The Journal of antimicrobial chemotherapy 2002; 49:185-8; Herbert et al., Antimicrobial agents and chemotherapy 1996; 40:2296-9). Therefore, a compound that induces the multiplication of mycobacteria cells in a nutrient-limited environment and in biofilms might be a potential candidate to increase the efficacy of bactericidal antibiotics on drug-tolerant cells.

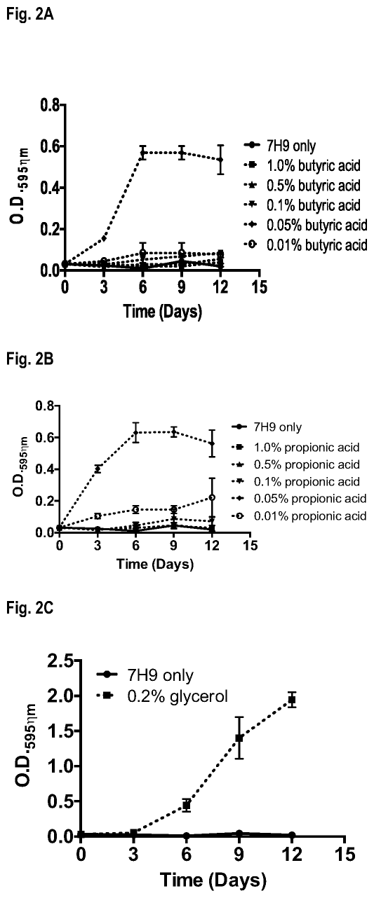

We determined the optimal concentration for the growth of MAH104 in non-supplemented 7H9 media since the amounts of these metabolites available in Biolog plates are unknown. For propionic acid and butyric acid, several concentrations were tested (1%, 0.5%, 0.1%, 0.05% and 0.01%), while for glycerol, the concentration was 0.2%. The experiments were made in non-supplemented 7H9 medium (without any carbon sources: no OADC, no glycerol, and no tween 20 or 80) (FIG. 2A-C), which is the condition applied in the biofilms assay. Results revealed that planktonic cultures of MAH104 were able to grow in non-supplemented 7H9 only when propionic acid (FIG. 2B) and butyric acid (FIG. 2A) were at 0.05%. As expected, glycerol was able to support MAH104 growth in non-supplemented 7H9 and MAH104 did not multiply in non-supplemented 7H9 (FIG. 2C).

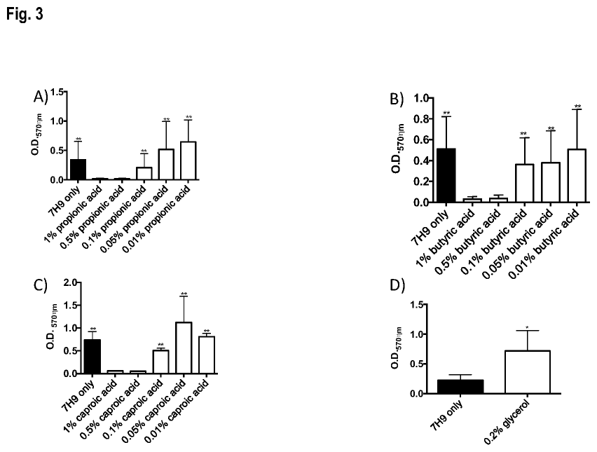

After determining the optimal concentration of propionic acid and butyric acid in non-supplemented 7H9, the capacity of these metabolites and glycerol to induce the growth of sessile forms of MAH104 in a nutrient-limited media was tested. Low mycobacterial cells density (10⁶/ml) resuspended in non-supplemented 7H9 media were inoculated in 96-well plates and incubated for 7 days at 37° C. to allow the bacteria to attach into polystyrene. The supernatant was discarded to remove planktonic cells and pre-attached bacterium was incubated for 7 days in nutrient-limited media (non-supplemented 7H9). Subsequently, these sessile MAH104 cells were incubated for 7 days at 37° C. with 0.2% glycerol or with 0.05% of fatty acids (propionic acid and butyric acid) in non-supplemented 7H9. The growth of sessile mycobacteria was followed by O.D._{595 nm} measurement and biofilm formation was determined through crystal violet (O.D._{570 nm}), since the multiplication of bacteria cells is linked with the formation of biofilms. No growth was observed when sessile MAH104 cells were cultivated in non-supplemented 7H9 broth. On the other hand, propionic acid, butyric acid and glycerol supported the growth of sessile forms. It is also important to highlight that the O.D._{570 nm} value for sessile MAH104 forms incubated with non-supplemented 7H9 (O.D._{570 nm}=0.11±0.039) had no significant difference in comparison with the blank (O.D._{570 nm}=0.091±0.007) (P=0.37), showing no biofilm formation. On the other hand, propionic acid, butyric acid and glycerol supported the growth of sessile forms. Moreover, the O.D._{570 nm} values of sessile MAH104 incubated with our tested substrates (propionic acid, O.D._{570 nm}=0.25±0.01; butyric acid, O.D._{570 nm}=0.32±0.02; caproic acid, O.D._{570 nm}=0.177±0.017) were significantly higher than the O.D._{570 nm} value from blank (O.D._{570 nm}=0.091±0.007) (P<0.01). FIG. 3A-D.

### Example 3: Glycerol but not Short-Chain Fatty Acids Promote the Growth of Mycobacteria Cells in Biofilms

The next step was to evaluate the capacity of tested substrates to promote the growth of mycobacteria cells that are present in biofilms. Static biofilm was established in the presence of glycerol and several concentrations of butyric acid and propionic acid (1%, 0.5%, 0.1%, 0.05%, 0.01%). MAH 104 cells in the presence of non-supplemented 7H9 were used as a negative control. A significant increase in the biofilm formation was observed when MAH104 was incubated with glycerol (3.2× higher) in comparison with biofilms incubated only in non-supplemented 7H9. Interestingly, mycobacteria incubated with 1% and 0.5% of propionic acid were unable to form biofilm and same results were obtained with butyric acid. In contrast, biofilm formation occurred in the presence of lower concentration of the short-chain fatty acids but we did not find any significant differences when compared to biofilm formed only in 7H9.

Next, the effect of short-chain fatty acids and glycerol in pre-established biofilms was tested. In parallel, the O.D._{595 nm} of pre-established biofilm cultures were also measured for 7 days in order to check whether the bacterial cells in biofilm multiplied in the presence of the targeted substrates. In the presence of glycerol, MAH104 cells displayed a significant increase in the O.D._{595 nm} values in comparison with the O.D._{595 nm} values from MAH104 biofilms cultivated only in 7H9 even after 24 hours of culture (glycerol, O.D._{595 nm}=0.609±0.069; 7H9 media, O.D._{595 nm}=0.452±0.049; P=0.026). In addition, we observed a significant increase in the biofilm formation (O.D._{570 nm} values) when mycobacteria biofilm was incubated with glycerol (7H9, O.D._{570 nm}=0.332±0.073; glycerol, O.D._{570 nm}=1.562±0.1751; P=0.0029). Furthermore, pre-established biofilms incubated with glycerol exhibited a higher biofilm formation than the established biofilms that were incubated with butyric acid, and propionic acid (glycerol, O.D._{570 nm}=1.562±0.1751; propionic acid, O.D._{570 nm}=0.3855±0.048; butyric acid, O.D._{570 nm}=0.490±0.121; O.D._{570 nm}=0.383±0.065). Altogether, these results suggest that glycerol but not short-chain fatty acids promote the multiplication of mycobacteria cells in biofilms.

### Example 4: Incubation with Glycerol and Short-Chain Fatty Acids Increase the Efficacy of Bactericidal Antibiotics Against Mycobacteria in Biofilms and in Macrophages

The studies disclosed here demonstrate that glycerol and short-chain fatty acids are used as an energy source by mycobacteria biofilms, and also can promote division of mycobacteria cells in nutrient-limited media. In addition, we observed that glycerol might also induce the multiplication of MAH104 cells in biofilms. Altogether, the current results indicate that short-chain fatty acids and glycerol increase the metabolic rate of mycobacteria cells in conditions that induce the emergence of drug-tolerant cells. Consequently, these metabolites might increase the susceptibility of mycobacteria in nutrient-limited media and in biofilms to bactericidal antibiotics. We then assessed the capacity of glycerol, propionic acid and butyric acid to enhance the efficacy of clarithromycin and amikacin against MAH104 cells in biofilms and inside macrophages.

Mycobacteria biofilms were first established and then treated or not treated with antibiotics. In parallel, biofilms were also co-treated with antibiotics and the targeted metabolites. Table 2 shows that the numbers of bacteria in biofilms treated with propionic acid and clarithromycin reduced about 20,000× in comparison with biofilms incubated with only clarithromycin (clarithromycin only, 8.9±0.8×10⁷; clarithromycin+propionic acid, 2.9±0.3×10³; P<0.05). Similar data were recorded for the co-treatment with amikacin and propionic acid (reduction of 24,722.22×) (amikacin only, 5.8±0.5×10⁷; amikacin+propionic acid, 3.6±0.6×10³; P<0.05). With respect to butyric acid, the co-treatment of biofilms with clarithromycin and butyric acid also decreased the numbers of mycobacteria more than biofilms treated with clarithromycin (clarithromycin only, 8.9±0.8×10⁷; clarithromycin+butyric acid, 2.7±×10³; P<0.05).

TABLE 2
In vitro antibiotic efficacy against MAH (strain 104)
in established biofilms supplemented
with glycerol and short-chain acids.

| Treatment | CFU/ml at 14 days |
|---|---|
| None | 4.2 +/− 0.5 × 10⁸ |
| Clarithromycin (16 μg/ml) | 8.9 +/− 0.8 × 10^{7*(1)} |
| Amikacin (4 μg/ml) | 5.8 +/− 0.5 × 10^{7*(1)} |
| Glycerol | 1.1 +/− 0.3 × 10^{9*} |
| Glycerol + clarithromycin | 1.7 +/− 0.2 × 10^{4* (1,2,3)} |
| Glycerol + amikacin | 7.0 +/− 0.2 × 10^{5*(1,2,3)} |
| Propionic acid | 5.5 +/− 0.4 × 10^{6*(1)} |
| Propionic acid + clarithromycin | 2.9 +/− 0.3 × 10^{3(1,2,3)} |
| Propionic acid + amikacin | 3.6 +/− 0.6 × 10^{3*(1,2,3)} |
| Butyric acid | 3.4 +/− 0.6 × 10^{6* (1)} |
| Butyric acid + clarithromycin | 2.7 +/− 0.5 × 10^{3*(1,2,3)} |
| Butyric acid + amikacin | 6.3 +/− 0.7 × 10^{3 (1,2,3)} |
| Caproic acid | 6.4 +/− 0.2 × 10^{6*(1)} |
| Caproic acid + clarithromycin | 4.9 +/− 0.6 × 10^{3*(1,2,3)} |
| Caproic acid + amikacin | 7.3 +/− 0.4 × 10^{3*(1,2,3)} |

⁽¹⁾P < 0.05 compared with control
⁽²⁾P < 0.05 compared with amikacin or clarithromycin
⁽³⁾P < 0.05 compared with short-chain fatty acids

Next, we evaluated whether propionic acid, butyric acid and glycerol could increase the bacterial killing by amikacin and clarithromycin since there is evidence that mycobacteria display tolerance to antibiotics when residing in macrophages (Adams et al., Cell 2011, 145:39-53; Rojony et al., Clinical proteomics 2019; 16:39). Table 3 below shows that a population of mycobacteria cells survived after two hours or four days of antibiotics treatment when inside THP1 cell line macrophages (non-treated, 8.2±0.3×10⁵ CFU/ml; clarithromycin, 3.9±0.3×10⁴ CFU/ml; amikacin, 4.8±0.5×10⁴ CFU/ml). Interestingly, the amount of mycobacteria killed increased significantly after co-treatment with propionic acid and the antibiotics (clarithromycin and amikacin). Also, the data indicated that butyric acid might also enhance the efficacy of clarithromycin and amikacin (P<0.01).

TABLE 3
Response of intracellular MAH to treatment of
macrophages with short-chain fatty acids and
amikacin or clarithromycin.

| | | CFU/ml of macrophage lysate | |
|---|---|---|---|
| MAH 104 | Treatment | 2 h | 4 days |
| MAH | None | 2.0 +/− 0.4 × 10⁵ | 8.2 +/− 0.3 × 10⁵ |
| MAH | butyric acid | 2.0 +/− 0.4 × 10⁵ | 8.4 +/− 0.5 × 10⁵ |
| MAH | clari + butyric | 2.0 +/− 0.4 × 10⁵ | 2.0 +/− 0.4 × 10^{4* (1,2,3)} |
| | acid | | |
| MAH | AK + butyric | 2.0 +/− 0.4 × 10⁵ | 2.0 +/− 0.3 × 10^{4* (1,2,3)} |
| | acid | | |
| MAH | propionic acid | 2.0 +/− 0.4 × 10⁵ | 8.0 +/− 0.2 × 10⁵ |
| MAH | clari + propionic | 2.0 +/− 0.4 × 10⁵ | 1.1 +/− 0.3 × 10^{4* (1,2,3)} |
| | acid | | |
| MAH | AK + propionic | 2.0 +/− 0.4 × 10⁵ | 1.7 +/− 0.3 × 10^{4* (1,2,3)} |
| | acid | | |
| MAH | caproic acid | 3.1 +/− 0.4 × 10⁵ | 5.3 +/− 0.4 × 10^{5* (1)} |
| MAH | clari + caproic | 3.1 +/− 0.4 × 10⁵ | 3.6 +/− 0.2 × 10^{4* (1,2,3)} |
| | acid | | |
| MAH | AK + caproic | 3.1 +/− 0.4 × 10⁵ | 1.6 +/− 0.4 × 10^{4* (1,2,3)} |
| | acid | | |
| MAH | glycerol | 3.1 +/− 0.4 × 10⁵ | 2.0 +/− 0.5 × 10^{5* (1)} |
| MAH | clari + glycerol | 3.1 +/− 0.4 × 10⁵ | 8.3 +/− 0.4 × 10^{3* (1,2,3)} |
| MAH | AK + glycerol | 3.1 +/− 0.4 × 10⁵ | 9.1 +/− 0.5 × 10^{3* (1,2,3)} |
| MAH | clarithromycin | 4.5 +/− 0.4 × 10⁵ | 3.9 +/− 0.3 × 10^{4* (1)} |
| | (16 μg/ml) | | |
| MAH | amikacin | 4.5 +/− 0.4 × 10⁵ | 4.8 +/− 0.5 × 10^{4* (1)} |
| | (4 μg/ml) | | |

⁽¹⁾ P < 0.05 compared with control
⁽²⁾ P < 0.05 compared with antibiotics
⁽³⁾ P < 0.05 compared with short-chain fatty acid

Biofilms were established from an MAH strain isolated from the lungs of a patient (MAH 3388). The activity of propionic acid and butyric acid to potentiate Amikacin activity was confirmed.

TABLE 4
Effect of amikacin treatment, alone or in combination
with butyric acid or propionic acid on
MAH biofilm established with MAH isolated from lungs.

| Strains | Antibiotics (4) | Short-chain FA | CFU/mL |
|---|---|---|---|
| MAH 3388 | — | — | 9.4 +/− 0.2 × 10⁷ |
| MAH 3388 | Amikacin | — | 5.4 +/− 0.9 × 10^{7*(1)} |
| MAH 3388 | — | propionic acid | 9.6 +/− 0.4 × 10⁷ |
| MAH 3388 | Amikacin | propionic acid | 3.1 +/− 0.5 × 10^{3*(1,2,3)} |
| MAH 3388 | — | butyric acid | 9.7 +/− 0.6 × 10⁷ |
| MAH 3388 | Amikacin | butyric acid | 2.4 +/− 0.5 × 10^{3*(1,2,3)} |
| MAH 3393 | — | — | 1.0 +/− 0.8 × 10⁸ |
| MAH 3393 | Amikacin | — | 5.2 +/− 0.6 × 10^{7*(1)} |
| MAH 3393 | — | propionic acid | 1.7 +/− 0.4 × 10⁷ |
| MAH 3393 | Amikacin | propionic acid | 1.6 +/− 0.5 × 10^{3*(1,2,3)} |
| MAH 3393 | — | butyric acid | 6.2 +/− 0.7 × 10⁷ |
| MAH 3393 | Amikacin | butyric acid | 3.1 +/− 0.2 × 10^{3*(1,2,3)} |

⁽¹⁾P < 0.05 compared with untried control
⁽²⁾P < 0.05 compared with propionic or butyric controls
⁽³⁾P < 0.05 compared with amikacin control
(4) amikacin concentration: 4 mg/ml

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### INCORPORATION BY REFERENCE

All patents and publications referenced herein are hereby incorporated by reference in their entireties.

1. A method for treating nontuberculous mycobacterium (NTM) infection in a patient, the method comprising administering to the patient one or more antibiotics, and administering a potentiator composition to the lungs of the patient.

2. The method of claim 1, wherein the potentiator composition comprises one or more metabolites selected from metabolites of the Kreb's cycle, a metabolite of β-oxidation pathway, a metabolite of lipid catabolism, an alkanoic acid or alkanoate, and glycerol.

3. The method of claim 1, wherein the potentiator composition comprises an aliphatic mono- or di-carboxylic acid, or a salt or ester thereof.

4. The method of claim 3, wherein the aliphatic mono- or di-carboxylic acid, or salt or ester thereof, comprises up to 16 carbon atoms.

5. The method of claim 4, wherein the aliphatic mono- or di-carboxylic acid, or salt or ester thereof, comprises up to 10 carbon atoms.

6. The method of claim 4, wherein the aliphatic mono- or di-carboxylic acid is a straight or branched chain fatty acid, or a salt or ester thereof.

7. The method of claim 6, wherein the straight or branched chain fatty acid is a short chain fatty acid, or a salt or ester thereof; and which is optionally an alkyl ester, and which is optionally a methyl or ethyl ester.

8. The method of claim 1, wherein the potentiator composition comprises one or more of: propanoic acid, or salt or ester thereof; butanoic acid, or salt or ester thereof; 2-methylpropanoic acid, or salt or ester thereof; pentanoic acid, or salt or ester thereof; 3-methylbutanoic acid, or salt of ester thereof; caproic acid, 4-methylpentanoic acid, or salt or ester thereof; sebacic acid, or salt or ester thereof; and pyruvic acid, or salt or ester thereof.

9. The method of any one of claims 1 to 8, wherein the potentiator composition comprises glycerol.

10. The method of any one of claims 1 to 9, wherein the potentiator composition is administered as a powder or aerosol for inhalation.

11. The method of claim 10, wherein the potentiator composition is administered by nebulizer.

12. The method of claim 11, wherein the potentiator composition comprises liposomes.

13. The method of any one of claims 1 to 12, wherein the patient is administered one or more antibiotics selected from: an aminoglycoside antibiotic, a macrolide antibiotic, ethambutol, and a rifamycin.

14. The method of claim 13, wherein the patient is administered an aminoglycoside antibiotic selected from amikacin, streptomycin, tobramycin, apramycin, arbekacin, astromicin, capreomycin, dibekacin, framycetin, gentamicin, hygromycin B, isepamicin, kanamycin, neomycin, netilmicin, paromomycin, rhodestreptomycin, ribostamycin, sisomicin, spectinomycin, and verdamicin, or a pharmaceutically acceptable salt thereof.

15. The method of claim 14, wherein the patient is administered amikacin or streptomycin or a pharmaceutically acceptable salt thereof.

16. The method of claim 14 or 15, wherein the aminoglycoside is administered locally to the lungs, and is optionally a powder formulation or nebulized formulation of amikacin.

17. The method of claim 16, wherein the aminoglycoside formulation is an aqueous solution or suspension delivered by a nebulizer.

18. The method of claim 17, wherein the aminoglycoside formulation is a liposomal formulation, which is optionally of amikacin.

19. The method of any one of claims 16 to 18, wherein the aminoglycoside is coformulated in the potentiator composition.

20. The method of any one of claims 13 to 19, wherein the patient is administered a macrolide antibiotic.

21. The method of claim 20, wherein the macrolide is selected from azithromycin, clarithromycin, erythromycin, fidaxomicin, carbomycin A, josamycin, kitasamycin, midecamycin acetate, oleandomycin, solithromycin, spiramycin, troleandomycin, tylosin, tylocine, and roxithromycin or a pharmaceutically acceptable salt thereof.

22. The method of claim 21, wherein the macrolide is administered orally, and is optionally selected from azithromycin or clarithromycin.

23. The method of any one of claims 13 to 22, wherein the patient is administered rifampin or rifabutin.

24. The method of claim 23, wherein the rifampin is administered orally.

25. The method of any one of claims 13 to 24, wherein the patient is administered ethambutol.

26. The method of claim 25, wherein the ethambutol is administered orally.

27. The method of any one of claims 1 to 26, wherein the non-tuberculous mycobacterial infection involves M. avium, M. avium subsp. hominissuis (MAH), M. abscessus, M. chelonae, M. bolletii, M. kansasii, M. ulcerans, M. avium complex (MAC) (M. avium and M. intracellulare), M. chimaera, M. conspicuum, M. peregrinum, M. immunogenum, M. xenopi, M. marinum, M. malmoense, M. mucogenicum, M. nonchromogenicum, M. scrofulaceum, M. simiae, M. smegmatis, M. szulgai, M. terrae, M. terrae complex, M. haemophilum, M. genavense, M. gordonae, M. fortuitum, M. fortuitum complex (M. fortuitum and M. chelonae), or a combination thereof.

28. The method of any one of claims 1 to 27, wherein the potentiator composition is administered at least three times weekly.

29. The method of claim 28, wherein the potentiator composition is administered once or twice daily.

30. The method of claim 28 or 29, wherein the administration period is at least 6 months.

31. The method of claim 30, wherein the administration period is at least 12 months, or at least 18 months.

32. The method of claim 30, wherein the administration period is less than one year.

33. A unit dose formulation for delivery by nebulizer, the formulation comprising: from 100 to 600 mg of an aminoglycoside antibiotic or a salt thereof, and effective amount of an aliphatic mono- or di-carboxylic acid, or a salt or ester thereof, to potentiate the aminoglycoside activity against nontuberculous mycobacterium (NTM).

34. The unit dose of claim 33, wherein the aliphatic mono- or di-carboxylic acid, or salt or ester thereof, comprises up to 16 carbon atoms.

35. The unit dose of claim 34, wherein the aliphatic mono- or di-carboxylic acid, or salt or ester thereof, comprises up to 10 carbon atoms.

36. The unit dose of claim 34, wherein the aliphatic mono- or di-carboxylic acid is a straight or branched chain fatty acid, or a salt or ester thereof.

37. The unit dose of claim 36, wherein the straight or branched chain fatty acid is a short chain fatty acid, or a salt or ester thereof; and which is optionally an alkyl ester, and which is optionally a methyl or ethyl ester.

38. The unit dose of claim 33, wherein the aliphatic mono- or di-carboxylic acid comprises one or more of: propanoic acid, or salt or ester thereof; butanoic acid, or salt or ester thereof; 2-methylpropanoic acid, or salt or ester thereof; pentanoic acid, or salt or ester thereof; 3-methylbutanoic acid, or salt of ester thereof; caproic acid, 4-methylpentanoic acid, or salt or ester thereof; sebacic acid, or salt or ester thereof; and pyruvic acid, or salt or ester thereof.

39. The unit dose of any one of claims 33 to 38, wherein the unit dose further comprises glycerol.

40. The unit dose of any one of claims 33 to 39, wherein aminoglycoside antibiotic is amikacin, and the amikacin is comprised in liposomes with the aliphatic mono- or di-carboxylic acid, or salt or ester thereof.
